(19) United States
(12) Patent Application Publication
(10) Pub. No.: US 2022/0259621 A1
(43) Pub. Date: Aug. 18, 2022

(54) NANOPARTICLE BIOHYBRID COMPLEXES

(71) Applicants: Alliance for Sustainable Energy, LLC, Golden, CO (US); Utah State University, Logan, UT (US); Montana State University, Bozeman, MT (US); The Regents of the University of Colorado, a body corporate, Denver, CO (US)

(72) Inventors: Paul Wayne KING, Golden, CO (US); Katherine Alice BROWN, Lakewood, CO (US); Lance C. SEEFELDT, Providence, UT (US); John W. PETERS, Bozeman, MT (US); Gordana DUKOVIC, Boulder, CO (US)

(21) Appl. No.: 17/734,809

(22) Filed: May 2, 2022

Related U.S. Application Data
(62) Division of application No. 15/818,450, filed on Nov. 20, 2017.
(60) Provisional application No. 62/423,891, filed on Nov. 18, 2016.

(51) Int. Cl. C12P 3/00; C12N 9/02; B82Y 5/00

(52) U.S. Cl. CPC C12P 3/00; B82Y 5/00; C12N 9/0095; C12Y 118/06001

(57) ABSTRACT

Disclosed herein are biohybrid protein complexes capable of using light energy to photocatalyze the reduction of N₂ into NH₃. Also provided are methods of using biohybrid protein complexes to enzymatically reduce N₂ to NH₃ using light rather than chemical energy as the driving force. These methods may also include the production and isolation of ammonia, hydrogen or both.

### CROSS-REFERENCE TO RELATED APPLICATIONS

This application claims priority under 35 U.S.C. 121 to, and is a divisional application of, U.S. application Ser. No. 15/818,450 filed on 20 Nov. 2017 which claims the benefit of U.S. Provisional Application No. 62/423,891, filed Nov. 18, 2016, the contents of which are incorporated herein by reference in its entirety.

### CONTRACTUAL ORIGIN

The United States Government has rights in this invention under Contract No. DE-AC36-08GO28308 between the United States Department of Energy and the Alliance for Sustainable Energy, LLC, the Manager and Operator of the National Renewable Energy Laboratory. This invention was made with government support under grant number DE-SC0010334 awarded by the Department of Energy. This invention was made with government support under grant number DE-SC0012518 awarded by the Department of Energy. The government has certain rights in the invention.

### BACKGROUND

The reduction of dinitrogen (N₂) to ammonia (NH₃) is a kinetically complex and energetically challenging multistep reaction that makes up the single largest input of fixed nitrogen (N) into the global biogeochemical cycle. Although the overall reaction releases energy, the cleavage of the nitrogen-nitrogen triple bond has a very large activation barrier. In the industrial Haber-Bosch process, NH₃ is produced via a dissociative reaction involving co-activation of dihydrogen (H₂) and N₂ over a Fe-based catalyst. The H₂ used for the reaction is produced by steam reforming of natural gas and results in co-production of significant amounts of CO₂. The energy required (>600 kJ mol⁻¹ NH₃) to achieve the high temperatures (500° C.) and pressures (200 atm) necessary to drive the reaction is also largely derived from fossil fuels.

In addition to its use in chemical fertilizers, ammonia also offers a means to store energy that can then be used to power an ammonia fuel cell. Currently, there is high interest in storing solar energy in the form of biofuels or reduced chemicals like ammonia, and using these products as energy carriers to power vehicles and fuel cell devices. Meeting the global demand for ammonia in a more energy-efficient and sustainable manner would lower the impact of current commercial processes on the environment (e.g., require less energy input and less carbon dioxide emissions) and would reduce dependence on fossil fuels.

The foregoing examples of the related art and limitations related therewith are intended to be illustrative and not exclusive. Other limitations of the related art will become apparent to those of skill in the art upon a reading of the specification and a study of the drawings.

### SUMMARY

In an aspect, a biohybrid complex is disclosed having a photoactive nanoparticle and an enzyme, wherein the photoactive nanoparticle produces electrons when exposed to light and the enzyme uses the electrons produced by the photoactive nanoparticle to catalyze an enzymatic reaction. In an embodiment, the biohybrid complex has an electron donor. In another embodiment, the electron donor is HEPES. In an embodiment, the light has a wavelength of from about 380 nm to about 450 nm. In yet another embodiment, the intensity of the light at the biohybrid complex is from about 1.8 mW cm⁻² to about 25 mW cm⁻². In an embodiment, the photoactive nanoparticle contains nanoparticles. In yet another embodiment, the photoactive nanoparticles are CdS nanoparticles. In an embodiment, the enzyme is a nitrogenase. In an embodiment, the nitrogenase is MoFe protein. In an embodiment, the enzymatic reaction produces up to about 86 mol NH₃ mol MoFe protein⁻¹ min⁻¹. In another embodiment, the enzymatic reaction produces up to about 827 mol H₂ mol MoFe protein⁻¹ min⁻¹. In yet another embodiment, the enzymatic reaction produces up to about 12000 mol NH₃ mol MoFe protein⁻¹ over about 300 minutes of exposure to light. In an embodiment, the enzymatic reaction produces up to about 120000 mol H₂ mol MoFe protein⁻¹ over about 300 minutes of exposure to light. In an embodiment, the photoactive nanoparticles are CdS nanoparticles and the enzyme is MoFe protein.

In an aspect, a method of producing ammonia is disclosed having the steps of contacting a nitrogenase biohybrid complex with nitrogen; exposing the nitrogenase biohybrid complex to light to generate ammonia; and isolating the generated ammonia. In an embodiment, the light has a wavelength from about 380 nm to about 450 nm. In another embodiment, the intensity of the light at the biohybrid complex is from about 1.8 mW cm⁻² to about 25 mW cm⁻². In an embodiment, the biohybrid complex has CdS nanoparticles. In another embodiment, the isolated ammonia is about 86 mol NH₃ mol biohybrid complex⁻¹ min⁻¹. In yet another embodiment, the isolated ammonia is about 12000 mol NH₃ mol biohybrid complex⁻¹ after about 300 minutes of exposure to light.

### BRIEF DESCRIPTION OF THE DRAWINGS

Exemplary embodiments are illustrated in referenced figures of the drawings. It is intended that the embodiments and figures disclosed herein are to be considered illustrative rather than limiting.

FIG. 1 depicts a reaction scheme for N₂ reduction by nitrogenase and CdS:MoFe protein biohybrids. Panel A shows the reduction of N₂ to NH₃ catalyzed by nitrogenase Fe protein. Panel B shows the reaction catalyzed by CdS:MoFe protein biohybrids.

FIG. 2 depicts photochemical reduction of N₂ to NH₃ by CdS:MoFe protein biohybrids.

FIG. 3 shows (panels A and B) TEM images of CdS nanocrystals with average dimensions of 38±5 Å (d)×168±16 Å (1) (Mean of N=200 measurements, ±SD) and (panel C) UV-vis spectrum of the CdS nanocrystals (black plot) overlaid with the emission spectrum of the 405 nm diode light source (gray plot).

FIG. 4 depicts (panel A) a calibration curve for the colorimetric NH₃ assay and (panel B) a calibration curve for the o-phthalaldehyde colorimetric NH₃ assay.

FIG. 5 depicts photochemical H₂ production by CdS:MoFe protein biohybrids. Panel (a) shows a time course of H₂ production by CdS:MoFe protein biohybrids (circles) and CdS:apo-MoFe protein biohybrids (squares). Panel (b) depicts the effects of addition of MoFe protein inhibitors on the turn over frequency (TOF) of H₂ production by CdS:MoFe protein biohybrids.

### DETAILED DESCRIPTION

Disclosed herein are biohybrid protein complexes capable of using light energy to photocatalyze the reduction of N₂ into NH₃. Also provided are methods of using biohybrid protein complexes to enzymatically reduce N₂ to NH₃ using light rather than chemical energy as the driving force. These methods may also include the production and isolation of ammonia (NH₃), hydrogen (H₂) or both. For example, CdS nanocrystals can be used to photosensitize the nitrogenase MoFe protein, allowing light harvesting to replace ATP hydrolysis to drive the enzymatic reduction of N₂ into NH₃. In certain embodiments, the turnover rate may be 75 min⁻¹, 63% of the ATP-coupled reaction rate for the nitrogenase complex under optimal conditions. CdS:MoFe protein biohybrids thus provide an example of a photochemical model for achieving light-driven N₂ reduction to NH₃.

The splitting of dinitrogen (N₂) and reduction to ammonia (NH₃) is a kinetically complex and energetically challenging multistep reaction. In the Haber-Bosch process, N₂ reduction is accomplished using high temperature and pressure, whereas N₂ fixation by the enzyme nitrogenase occurs under ambient conditions using chemical energy from ATP hydrolysis. The ability to create complexes between nanomaterials and nitrogenase and other enzymes allows photoexcited electrons to drive difficult catalytic transformations and provides new tools for mechanistic investigations. For example, biohybrid complexes can be used to examine how the flux and thermodynamics of photoexcited electron transfer influence the turnover and fidelity of catalytic product formation.

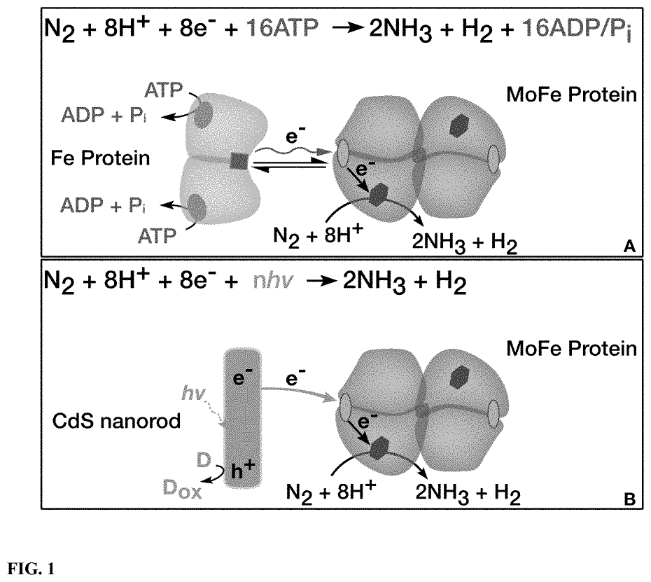

In nitrogen-fixing bacteria, the enzymatic reduction of N₂ to NH₃ is catalyzed by nitrogenase enzymes, and proceeds via the hydrogenation of N₂ through metal-hydride intermediates rather than from reaction with H₂. The Mo-dependent nitrogenase is a multi-protein complex composed of MoFe and Fe proteins, named after the metals in their active sites. Although nitrogenase functions at room temperature (25° C.) and pressure (1 atm), it requires a large input of chemical energy provided by the hydrolysis of ATP (FIG. 1, panel A). A minimum of 16 moles of ATP (ΔG°=−488 kJ mol⁻¹ or 5 eV mol⁻¹ of N₂ reduced) is required to reduce N₂ to NH₃. During catalysis, the Fe protein associates and dissociates from the MoFe protein resulting in the eight sequential electron transfer/ATP hydrolysis events required to generate one mole of NH₃. Reducing equivalents accumulate at the catalytic site FeMo cofactor (FeMo-co) as Fe-hydrides, which directly participate in conversion of N₂ to NH₃ with an obligatory stoichiometric reduction of two protons to make H₂ (FIG. 1, panel A).

The biohybrid complexes disclosed herein are capable of using light energy rather than chemical energy to catalyze enzymatic reactions. Biohybrid complexes include two principal components: an optically active (photoactive) nanoparticle/nanocrystal component that acts as a source of electrons when exposed to light energy and an enzyme component capable of utilizing the electrons produced by the nanocrystal. Biohybrid complexes may also include an electron donor component such as a buffer that can be readily replenished to provide a steady source of electrons.

The photoactive nanoparticle component may be a nanoscale material capable of generating electrons upon exposure to light energy. Exemplary materials include quantum dots, metal nanoparticles (e.g., those containing gold, silver, copper, etc.), or up-conversion nanoparticles comprising solid-state materials doped with rare-earth ions (e.g., lanthanide-doped nanoparticles such as NaYF₄ co-doped with Yb³⁺/Er³⁺ or Yb³⁺/Tm³⁺). Although CdS nanocrystals are exemplified herein, additional photoactive nanocrystals are also suitable for use in biohybrid complexes. Nanoparticles may be spheres, rods or other shapes, and typically have dimensions from about 1 nm to about 100 nm. For example, nanorods may have lengths from about 10 nm to about 100 nm and diameters from about 1 nm to about 10 nm.

Quantum dots are nanocrystals of a semiconductor material with diameters that are small enough, typically on the order of a few nanometers in size, such that their free charge carriers experience quantum confinement in all three dimensions. This allows quantum dot properties (band gap, absorption spectrum, etc.) to be highly tunable, as quantum dot size can be controlled during fabrication. Quantum dot materials include elemental or compound semiconductor, metal, or metal oxide nanocrystal material such as metal chalcogenides (e.g., PbS, PbSe, PbTe, CdSe, CdS, CdTe, CuInS, CuInSe, ZnS, ZnSe, ZnTe, HgTe, CdHgTe or combinations thereof), Group III-V materials (e.g., InP, InAs, GaAs Si, Ge, SiGe, Sn or combinations thereof), metal oxides (e.g., ZnO, MoO, TiO₂ or combinations thereof), or perovskite nanocrystals (e.g., CsPbBr₃, CsPbI₃, CsPbCl₃, CsSnI₃ or combinations thereof).

Low potential chemical donors or photoexcited chromophores can directly deliver electrons to the MoFe protein. Complexes between MoFe protein and the low potential donor Eu(II)-L or Ru-photosensitizers support the catalytic reduction of protons or non-physiological C or N substrates (e.g., C₂H₂, HCN, N₂H₄, N₃⁻). However, these complexes are unable to catalyze N₂ reduction, and rates for non-physiological substrates are low (up to 8.5 min⁻¹) compared to physiological reaction rates (e.g., 500 min⁻¹ for C₂H₂ reduction). In the case of Ru-photosensitizers, Ru conjugate can be unstable, resulting in the loss of photocatalytic rates and low quantum yields (QY≤1%).

Although CdS nanorods have a low photoexcited state potential (−0.8 V vs. NHE), other reductants, such as Eu(II)-L, have lower potentials (as low as −1.2 V vs. NHE), yet the CdS nanorods support N₂ reduction by MoFe protein. Without being bound by any particular theory, one possible explanation for the observations with CdS nanorods may be the rapid delivery of successive electrons possible due to strong light absorption by the CdS nanorods, which could allow achievement of the 4 electron reduced FeMo-co state (E4) that is required for N₂ binding and reduction. Slow accumulation of electrons (low e-flux) on FeMo-co in the presence of other (photo)chemical donors could allow less reduced FeMo-co states (e.g., E2) to oxidize by evolving H₂ before N₂ binds. It is also possible that the binding of the CdS nanorod to the MoFe protein could induce protein conformational changes necessary to achieve N₂ reduction that normally occur upon Fe protein binding.

The enzyme component of a biohybrid complex may be any enzyme capable of utilizing electrons to catalyze an enzymatic reaction (e.g., enzymes that use electrons and chemical energy sources such as ATP). Examples include enzymes involved in electron transport chains such as those responsible for oxidative phosphorylation, photosynthesis, or cellular respiration. Many types of oxidases, hydrogenases, reductases, dehydrogenases, catalases, or enzymes that require co-enzymes (e.g., nicotinamide/flavin adenine dinucleotides) are examples of suitable enzyme components. Specific examples include nitrogenase enzymes that reduce nitrogen to ammonia, such as the MoFe protein. The MoFe protein is a heterotetramer comprising iron-sulfur P-clusters that uses electrons to reduce N₂ to NH₃. Nitrogenases can be found in many bacterial species, including species of cyanobacteria, green sulfur bacteria, Azotobacter, Rhizobium, Spirillum, and Frankia.

Suitable enzymes may be derived from microorganisms such as bacteria, fungi, yeast or the like via cell lysis and isolation techniques, or produced recombinantly. Polypeptides may be retrieved, obtained, or used in “substantially pure” form, a purity that allows for the effective use of the protein in any method described herein or known in the art. For a protein to be most useful in any of the methods described herein or in any method utilizing enzymes of the types described herein, it is most often substantially free of contaminants, other proteins and/or chemicals that might interfere or that would interfere with its use in the method (e.g., that might interfere with enzyme activity), or that at least would be undesirable for inclusion with a protein.

The biohybrid complexes disclosed herein are capable of carrying out enzymatic reactions when exposed to light energy. Light energy may be provided by natural light sources such as sunlight or artificial light sources such as lamps (e.g., incandescent, fluorescent, or high-intensity discharge lamps), diodes, lasers, and sources of luminescence. Light sources tailored to provide light of a specified wavelength or energy level or range of wavelengths or energy levels may be used. In certain embodiments, a photoelectrochemical cell or device that under illumination generates electrical current may be coupled (wired) to an electrode that has a layer of nitrogenase that then catalyzes a nitrogen reduction reaction.

In certain embodiments, the biohybrid complexes or reactions being catalyzed by the biohybrid complexes may also comprise an electron donor. Typical electron donors will serve as sacrificial electron donors to facilitate the activities of the biohybrid complexes and can be readily replenished in a reaction. Examples include electron donating buffers (such as HEPES, MOPS, MES, Tris, ascorbic acid buffers, etc.), electron donating solvents, aromatic compounds, amine solvents, or catalysts that oxidize water.

Also provided are methods for reducing nitrogen to ammonia and hydrogen and isolating one or more of these products. Specific examples of using CdS/nitrogenase biohybrid complexes to generate ammonia and hydrogen from nitrogen are provided in the examples below. Biohybrid complexes may be exposed to nitrogen in a closed system, then illuminated with a light source to generate ammonia and hydrogen. Reaction products may then be separated by conventional means.

For example, biohybrid complexes may be placed in a reaction vessel fed with a source of nitrogen (e.g., pure nitrogen gas, air, or mixtures thereof) and illuminated with light. Gaseous hydrogen may be recovered from the head space of the reaction vessel and further processed to separate out gaseous ammonia and any impurities or unreacted gases. Liquid ammonia may likewise be removed from the vessel and further purified. Conventional methods of absorption, fractionation, distillation, and other means of altering temperatures and pressures to separate hydrogen, ammonia and other reaction components may be used to isolate and purify hydrogen and ammonia products.

### CdS Nanocrystal Synthesis

Cadmium sulfide (CdS) seeds were synthesized from an initial mixture of 0.100 g cadmium oxide (CdO, 99.99%, Aldrich), 0.603 g octadecylphosphonic acid (ODPA, 99%, PCI), and 3.299 g trioctylphosphine oxide (TOPO, 99%, Aldrich), which were degassed then heated to 300° C. under argon for 30 minutes to dissolve the CdO. The solution was cooled to 120° C., degassed for 30 minutes, then heated to 320° C. under argon. After the temperature stabilized, sulfur stock solution (0.179 g hexamethyldisilathiane ((TMS)2S, synthesis grade, Aldrich) in 3 g of tributylphosphine (TBP, 97%, Aldrich) was quickly injected. The nanocrystals were allowed to grow at 250° C. for 7.5 minutes, after which, the reaction was stopped by cooling and subsequently injecting toluene. The CdS seeds were precipitated with methanol. After transfer to the glovebox and washing with toluene/methanol (2×), the final product was dissolved in trioctylphosphine (TOP, 97%, Strem).

The CdS seeds had an absorbance peak at 408 nm, and the estimated molar absorptivity (F) of the CdS seeds was 3.96×105 cm⁻¹ M⁻¹ at 408 nm. To synthesize the rods, 0.086 g CdO, 3 g TOPO, 0.290 g ODPA, and 0.080 g hexylphosphonic acid (HPA, 99%, PCI) were degassed under vacuum at 120° C. The solution was heated to 350° C. under argon for 30 minutes then 1.5 mL of TOP was added. When the temperature of the Cd-containing solution stabilized at 350° C., the seed-containing solution (0.124 g of sulfur (S, 99.998%, Aldrich) in 1.5 mL of TOP mixed with 8×10-8 mol CdS QD seeds) was quickly injected. After an 8 minute reaction time, the particles were cooled, transferred to the glovebox, and precipitated with a 1:1:1 mixture of acetone, toluene, and methanol to prepare for cleaning. The nanocrystals were cleaned by first redissolving in toluene, washing with octylamine, and precipitation with methanol. The nanocrystals were then redissolved in chloroform, washed with nonanoic acid, and precipitated with ethanol. The resulting particles were redissolved in toluene.

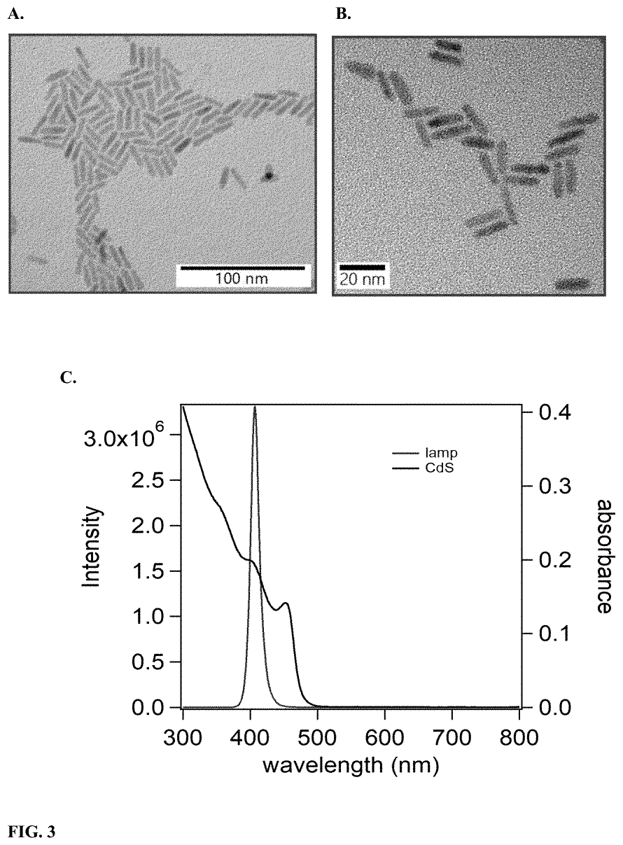

The CdS nanocrystals had an average diameter of 38±5 Å, and an average length of 168±16 Å as determined by measurements of 200 particles in transmission electron micrograph (TEM) images (FIG. 3, panels A and B). The F value of the CdS nanocrystals was determined by correlating absorption spectra with Cd²⁺ concentrations determined from elemental analysis by inductively coupled plasma optical emission spectroscopy (ICP-OES). The estimated 350 value of the CdS nanocrystals is 5.8×106 M⁻¹ cm⁻¹ based on a value of 1710 M⁻¹ cm⁻¹ per Cd²⁺ and an estimated number of Cd²⁺ per nanocrystal from the average nanocrystal dimensions.

### CdSe Nanocrystal Synthesis

For the preparation of CdSe nanocrystals capped with organic ligands, 4 g TOPO, 2.5 g hexadecylamine (HDA, 98%, Aldrich) and 0.075 g tetradecylphosphonic acid (TDPA, 99%, PCI) were dried and degassed under vacuum at 120° C. in a 25 mL three-neck flask. Under argon, 1 mL of a stock solution of Se precursor [0.79 g of selenium shot (99.99%, Aldrich) in 8.3 g of TOP] was added and the mixture was again dried and degassed under vacuum at 110° C. With the reaction temperature stabilized at 300° C. under argon, 1.5 mL of Cd precursor stock solution [0.12 g of cadmium acetate (99.999%, Strem) in 2.5 g of TOP] was quickly injected under vigorous stirring, resulting in nucleation of CdSe nanocrystals. The temperature was set to 260° C. for nanocrystal growth. Growth times of 0.3 minutes, 1.0 minute and 15 minutes were used to produce nanocrystals of varying diameters. After growth, the reaction mixture was cooled to 90° C. The mixture was added to a 20% (v/v) ethanol in chloroform solution and centrifuged to precipitate the nanocrystals. Under an inert atmosphere in a glovebox, the supernatant was discarded and the nanocrystals were redissolved in toluene. The solution was centrifuged to precipitate excess HDA. The resulting nanocrystals were washed with a 1:2 mixture of isopropanol:ethanol and redispersed in toluene. Nanoparticle diameters of 2.5, 2.7 and 3.4 nm were determined from the first excited state 1S3/2(h) to 1S(e) transition peak wavelength (515, 535 and 567 nm) as described in Yu et al., Chem. Mater. 15, 2854-2860 (2003).

### Nanocrystal Ligand Exchange

CdS and CdSe nanocrystals were solubilized in water by ligand exchange with mercaptopropionic acid (MPA). First, 1.27 mmol of 3-mercaptopropionic acid (3-MPA, Sigma Aldrich ≥99%) was dissolved in 20 mL of methanol. The solution pH was increased to 11 with tetramethylammonium hydroxidepentahydrate salt (Sigma Aldrich). A sample of nanocrystals was precipitated from toluene solution using methanol. The precipitated nanocrystals were then mixed with the MPA/methanol solution until the mixture was no longer cloudy. The water-soluble nanocrystals were precipitated with toluene. The resulting MPA-capped particles were dried under vacuum and dispersed in Tris buffer, pH 7.

### Transmission Electron Microscopy (TEM)

TEM sample grids were prepared by drop casting on carbon film, 300 mesh copper grids from Electron Microscopy Sciences. The image at the 100 nm scale was acquired with a FEI Tecnai Spirit BioTwin operating at 100 keV and equipped with a bottom mounted FEI Eagle 4K camera. The image at the 20 nm scale was acquired with a FEI Tecnai F-20 operating at 200 keV and equipped with a Gatan Ultrascan US-4000 camera. Lengths and diameters were determined from an average of 200 nanocrystals.

Azotobacter vinelandii Nitrogenase Purification

Azotobacter vinelandii strain DJ995 (wild type MoFe protein) and DJ1003 (apo-MoFe protein) was grown and the corresponding nitrogenase MoFe proteins, with a 7×His-tag near the carboxyl-terminal end of the α-subunit, were expressed and purified as described (Christiansen et al., Biochemistry 37, 12611-12623 (1998)). Protein concentrations were determined by the Biuret assay. The purities of these proteins were >95% based on SDS-PAGE analysis with Coomassie staining. Handling of proteins and buffers was done in septum-sealed serum vials under an argon atmosphere or on a Schlenk vacuum line. All liquids were transferred using gas-tight syringes. All reagents were obtained from Sigma Aldrich (St. Louis, Mo.) or Fisher Scientific (Fair Lawn, N.J.) and were used without further purification.

### Nanocrystal and Donor Optimization

Different nanocrystal:MoFe protein biohybrids were prepared under a 100% N₂ atmosphere by mixing individual solutions of 10 μM CdS or CdSe nanocrystals and 4.3 μM MoFe protein tetramer (1 mg mL⁻¹) to achieve a final molar ratio of 2:1 nanocrystal:MoFe protein tetramer. The mixtures were diluted into 50 mM Tris-HCl, 5 mM NaCl, pH 7, and 100 mM ascorbic acid to a final concentration of 200 nM nanocrystals and 100 nM MoFe protein tetramer and a final volume of 300 μL. Reactions were stirred for 30 minutes under illumination with a 405 nm diode light source (Ocean Optics) at 11 mW (˜1.8 mW cm-2 at the sample) in sealed vials with a total volume of 1.5 mL. The amount of H₂ produced was determined by gas chromatography (GC) on 0.2 mL of the headspace gas phase. Turnover frequencies (means of N=4 samples) were calculated as the total nmol of H₂ produced during the illumination time.

Donors were tested with CdS:MoFe protein biohybrids that were prepared under a 100% N₂ atmosphere by mixing individual solutions of 2.5 μM CdS and 2.13 μM MoFe protein tetramer (0.5 mg mL⁻¹) to achieve a final molar ratio of 1:1 CdS:MoFe protein tetramer. The mixtures were diluted into 50 mM Tris-HCl, 5 mM NaCl, pH 7, and the hole scavenger under investigation (HEPES, MES and MOPS at 500 mM, ascorbic acid at 100 mM, or Tris alone at 50 mM) to a final concentration of 16.7 nM CdS and MoFe protein and a final volume of 300 μL. Control reactions of CdS alone were prepared at a final concentration of 16.7 nM CdS in identical buffer conditions for each hole scavenger. Reactions were stirred for 30 minutes under illumination with a 405 nm diode light source (Ocean Optics) at 11 mW (˜1.8 mW cm⁻² at the sample) in sealed vials with a total volume of 1.5 mL.

Light-Driven NH₃ and H₂ Production Assays

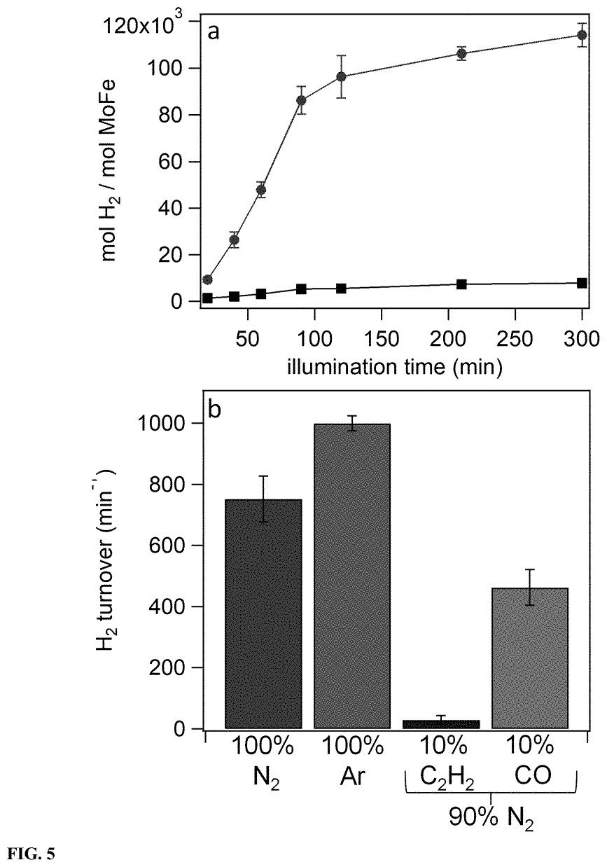

CdS:MoFe protein biohybrids were prepared under a 100% N₂ atmosphere by mixing individual solutions of 2.5 μM CdS and 2.13 μM MoFe protein tetramer (0.5 mg mL⁻¹) to achieve a final molar ratio of 1:1 CdS:MoFe protein tetramer. The mixtures were diluted into 500 mM HEPES, pH 7, to a final concentration of 16.7 nM CdS and MoFe protein and a final volume of 300 μL. Reactions were stirred under illumination with a 405 nm diode light source (Ocean Optics) at 25 mW cm⁻² (˜3.5 mW cm⁻² at the sample) in sealed vials with a total volume of 1.5 mL. The amount of NH3 produced was measured by colorimetric assay (BioVision), described in detail below. The amount of H₂ produced by CdS:MoFe protein biohybrids was determined by gas chromatography (GC) on 0.2 mL of the headspace gas phase. Reaction velocities (averages derived from 4 samples) were calculated as the total nmol of H₂ produced by each sample during the total illumination time (FIG. 5).

FIG. 5 (panel a) shows a time course of H₂ production by CdS:MoFe protein biohybrids (circles) and CdS:apo-MoFe protein biohybrids (squares). Reactions (16.7 nM CdS, 16.7 nM MoFe protein or 16.7 nM apo-MoFe protein, 500 mM HEPES, pH 7.0) were equilibrated under 100% N₂ stirred under illumination with 3.5 mW cm⁻² (at the sample) 405 nm light at 25° C. FIG. 5 (panel b) shows the effects of addition of MoFe protein inhibitors on the turn over frequency (TOF) of H₂ production by CdS:MoFe protein biohybrids. Reactions (16.7 nM CdS, 16.7 nM MoFe protein in 500 mM HEPES, pH 7.0 under 100% N₂ (N₂), 100% Argon (Ar), 90% N₂ with 10% of acetylene (C₂H₂), or 90% N₂ with 10% carbon monoxide (CO)) were stirred for 2 hours under illumination with 3.5 mW cm⁻² 405 nm light at 25° C. (Mean of N=4 independent measurements, ±SD).

### Colorimetric Assay of NH₃ Production

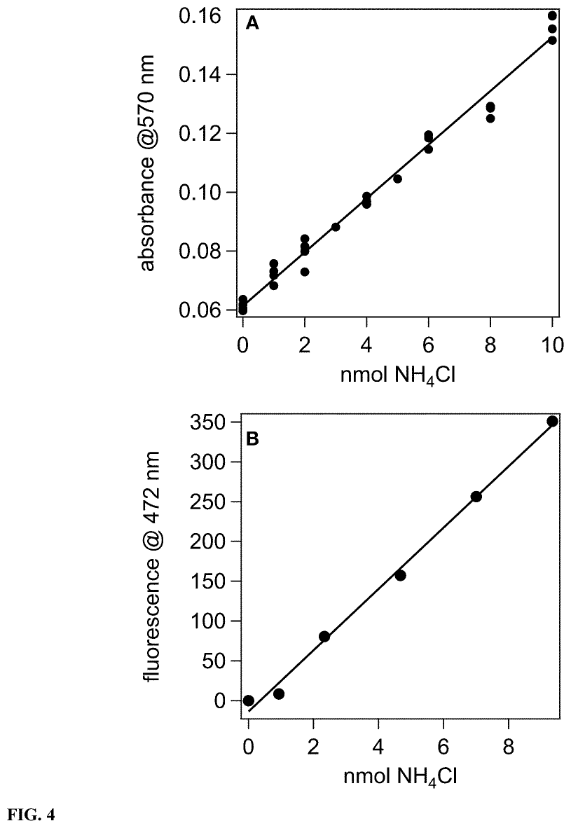

The amount of NH₃ produced was measured using a colorimetric ammonia assay kit (BioVision). Briefly, 50 μL of the CdS:MoFe protein reaction (total volume of 300 μL) was mixed with 50 μL of kit reaction buffer and incubated at 37° C. for 1 hour. Calibration curves were prepared from CdS nanocrystals (16.67 nM) that had been kept in the dark with the appropriate amount of ammonium chloride (FIG. 4, panel A). The presence of CdS in the kit shifted the baseline of the 570 nm absorbance signal but did not affect the slope of the A570 value vs. mol of NH₄Cl nor the linearity of the calibration curves. The sample absorbance at 570 nm was used to determine the amount of NH₃ present based on the calibration standards.

The calibration curve shown in FIG. 4 (panel A) was by adding ammonium chloride in the amount indicated on the x-axis to 50 μL of CdS nanoparticles (16.67 nM), then mixing with 50 μL of kit reaction buffer and incubated at 37° C. for 1 hour. The absorbance at 570 nm was measured by plate reader (Tecan Infinate M200 Pro). The line shows linear fit (y=a*x+b) of N=4 independent calibration curves (a=0.0091±0.0002, b=0.061±0.001; ±SD). The 570 nm absorbance value in the absence of added NH₄Cl (shown on the plot) is 0.0613±0.0012 (mean of N=4 measurements, ±SD).

FIG. 4 (panel B) shows the calibration curve for the o-phthalaldehyde colorimetric NH₃ assay. A solution of CdS:MoFe protein biohybrids (16.67 nM) in assay buffer were prepared, incubated in the dark for 90 minutes, then run through a 10 kDa spin concentrator (Corning Spin-X UF) at 14,000 rpm for 5 minutes to separate CdS:MoFe protein biohybrids. Ammonium chloride in the amount indicated on the x-axis was added to aliquots of the filtered solution to a volume of 50 μL. 1 mL of the o-phthalaldehyde solution was added and samples were incubated in the dark for 30 minutes at room temperature. The fluorescence (λexcitation/λemission 410 nm/472 nm) of the solutions was measured using a Shimadzu Model RF-5301 PC spectrofluorometer and the software provided with the instrument. The line shows linear fit (y=a*x+b) of the calibration curve (a=38.505, b=165.43).

### Biohybrid Photocatalysis

FIG. 1 illustrates a reaction scheme for N₂ reduction by nitrogenase and the CdS:MoFe protein biohybrids (panel A). The reduction of N₂ to NH₃ catalyzed by nitrogenase Fe protein (homodimer represented in green; MgATP binding site in orange spheres; [4Fe-4S] cluster brown square) and MoFe protein (α2β2 tetramer represented in gray and purple; FeMo-co, red hexagon; [8Fe-7S] P cluster, blue sphere). Hydrolysis of 16 ATP by Fe protein (Em=−0.42 V) is required for the sequential transfer (signified by the equilibrium arrow) of 8 electrons (e−) to MoFe protein (Em=−0.31 V) for catalytic reduction of N₂ to 2NH₃ and 1H₂. Panel B shows the reaction catalyzed by CdS:MoFe protein biohybrids (measured product ratios were 1NH₃/10H₂, with n≈98 absorbed photons). Under illumination, photon absorption (405 nm photon=3.06 eV) by CdS nanorods (orange; lowest energy transition, Eg=2.72 eV; FIG. 3) generates photoexcited electrons (E=−0.8 eV) and holes (E=+1.9 eV), where direct electron injection from CdS into MoFe protein (blue arrow) is thermodynamically favored (ΔE=0.5 V). The ground state of the CdS nanorod is regenerated by the oxidation of a sacrificial electron donor (D), such as HEPES (Eₘ=+0.8 V vs SHE).

N₂ reduction by the MoFe protein when it is adsorbed onto CdS nanocrystals to form biohybrid complexes was examined. Semiconductor nanocrystals are quantum confined materials with size-tunable photoexcited electron and hole energy levels. Different nanocrystalline materials were tested (Table 1) and CdS nanorods (d≈38±5 Å, 1≈168±16 Å; FIG. 3) were observed to deliver photogenerated electrons to the MoFe protein with the highest enzymatic turnover. The size, shape and surface electrostatics of the CdS nanorods complement the MoFe protein dimensions (d≈69 Å, 1≈110 Å) and surface electrostatics to support self-assembly into complexes. The lowest energy transition of the CdS nanorods is in the visible region of the solar spectrum (Eg=2.72 eV, λabsorption=456 nm, FIG. 3) and the reduction potential of the first photoexcited state transition, −0.8 V vs. NHE, is sufficiently negative to reduce the MoFe protein (−0.31 V) to drive electron transfer for catalytic reduction of N₂ to NH₃ (FIG. 1, panel B).

Table 1 depicts turnover frequencies (TOF) of H₂ production for 30 min illumination of MoFe protein with different nanocrystal materials and diameters.

TABLE 1

| | Nanocrystal | | |
|---|---|---|---|
| Nanocrystal | diameter | ^{a}TOF | ^{b}ε(M⁻¹ |
| material | (nm) | (s⁻¹) | cm⁻¹) |
| CdS nanorods | 3.8 | 6.2 ± 1.7 | 4.1 × 10⁶ |
| CdSe quantum | 2.5 | 1.5 ± 0.1 | 7.6 × 10⁴ |
| dots | 2.7 | 0.22 ± .04 | 1.4 × 10⁵ |
| | 3.4 | 0.19 ± .02 | 4.6 × 10⁵ |

^{a}Reactions were stirred under illumination with 405 nm diode light at ~1.8 mW cm⁻² at the
sample. Levels of H₂ were measured after 30 min by GC. Mean of N = 4 independent reactions, ± SD.
^{b}Calculated from the nanoparticle absorbance spectra and the established first excited state
1S3/2(h) → 1S(e) transition peak wavelength and extinction coefficient.

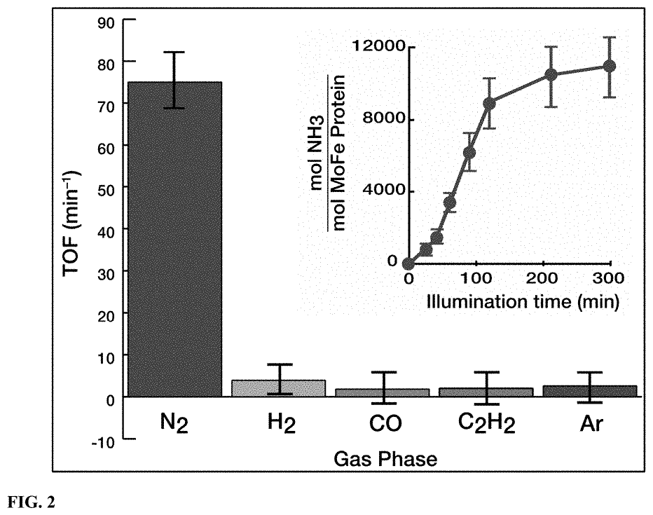

Photoexcitation of the CdS:MoFe protein biohybrids under a 100% N₂ atmosphere resulted in the direct light-driven reduction of N₂ to NH₃ (FIG. 2; FIG. 4; Tables 2-4). Transfer of low potential electrons to the MoFe protein from photoexcited CdS nanorod replaces ATP-coupled electron transfer by Fe protein. The reaction utilized a sacrificial electron donor, HEPES, which produced a high turnover over frequency (TOF) with a low background compared to other donors (Table 2). Control reactions that lacked a component (e.g., HEPES, CdS, light, or a functional MoFe protein) or utilized apo-MoFe protein that lacks FeMo-co did not reduce N₂ (Tables 3 and 5). Illumination under ˜3.5 mW cm⁻² of 405 nm light led to peak NH₃ production rates of 315±55 nmol NH₃ (mg MoFe protein)⁻¹ min⁻¹ at a TOF of 75 min⁻¹ (FIG. 2; Table 6). The values correspond to 63% of the NH₃ production (500 nmol NH₃ (mg MoFe protein)⁻¹ min-), and TOF (119 min⁻¹) catalyzed by the Fe protein and ATP-dependent reaction under optimal conditions (Table 6). The estimated QY of 3.5% for conversion of absorbed photons to NH₃ (QY=23.5% for the co-production of NH₃ and H₂; Tables 7 and 8) is higher than reported for other non-physiological reactions. N₂ reduction persisted for up to 5 hours under constant illumination (FIG. 2, inset; Tables 9 and 10) with a turnover number (TON) of 1.1×104 mol NH₃ (mol MoFe protein)⁻¹. This indicates that the MoFe protein in CdS:MoFe protein biohybrids is capable of functioning at rates comparable to physiological TOF by nitrogenase.

In FIG. 2, the TOF of catalytic reduction of N₂ to NH₃ was measured under 100% N₂ (N₂). The effects of MoFe protein inhibitors on the TOF are shown for 10% of either H₂ (H₂), carbon monoxide (CO), or acetylene (C₂H₂) in a bulk phase of 90% N₂. TOF for the CdS:MoFe protein biohybrids under 100% Argon (Ar) is shown as a negative control for comparison. Measured values were taken after 2 hours of illumination at 25° C. for reactions comprised of 1:1 molar ratios of CdS nanorods and MoFe protein tetramer. The data are means of N=4 independent measurements±SD calculated by standard error propagation. The inset shows the time course of NH₃ production by CdS:MoFe protein biohybrids under 100% N₂ (TON=1.1×104 mol NH₃ (mol MoFe protein)⁻¹; see Table 10).

The mechanism of N₂ reduction by the MoFe protein co-produces H₂ (FIG. 1), which was also observed as a co-product during CdS:MoFe protein photocatalytic N₂ reduction (FIG. 5; Tables 4 and 5). These data support a mechanism of N₂ reduction by the CdS:MoFe protein biohybrids that is analogous to the mechanism of MoFe protein:Fe protein catalysis. CdS inhibition of Fe protein dependent catalysis (Table 11) indicates CdS binds at or near the Fe protein binding site on MoFe protein (FIG. 1, panel B), however it is not known whether the P cluster serves as an intermediate in electron transfer during photocatalysis.

Table 2 depicts turnover frequencies for H₂ production by CdS:MoFe protein biohybrids with various hole scavengers.

TABLE 2

| | ^{b}nmol H₂ | ^{c}nmol H₂ | Corrected |
|---|---|---|---|
| ^{a}Hole | produced CdS: | produced | TOF |
| Scavenger | MoFe protein | CdS alone | (min⁻¹) |
| HEPES | 14.7 | 0.7 | 93.8 |
| MOPS | 13.1 | 1.5 | 76.9 |
| MES | 19.6 | 6.15 | 89.9 |
| Ascorbic Acid | 14.7 | 8.3 | 73.2 |
| Tris | ND | ND | — |

^{a}Donor concentrations: HEPES, MES, and MOPS, 500 mM; Ascorbic Acid, 100 mM; Tris, 50 mM.
^{b}16.7 nM CdS, 16.7 nM MoFe protein. Reactions were stirred for 30 min under illumination with
405 nm diode light at ~1.8 mW cm⁻². The levels of H₂ were measured by GC. Average of N = 2
independent reactions.
ND, not-detected.
^{c}16.7 nM CdS. Reactions were stirred for 30 min under illumination with 405 nm diode light at
~1.8 mW cm⁻². The levels of H₂ were measured by GC. Average of N = 2 independent reactions.

Table 3 depicts measurements of NH₃ produced by CdS:MoFe protein biohybrids by the colorimetric ammonia assay.

TABLE 3

| | | ^{b}Absorbance | ^{c}nmol NH₃ | ^{d}nmol NH₃ |
|---|---|---|---|---|
| ^{a}Sample | Gas Phase | 570 nm | in aliquot | in reaction |
| CdS:MoFe | 100% N₂ | 0.136 ± 0.005 | 8.2 ± 0.6 | 48.7 ± 3.4 |
| protein | 10% C₂H₂ | 0.069 ± 0.002 | 0.9 ± 0.3 | 5.2 ± 1.6 |
| | 90% N₂ | | | |
| | 10% CO | 0.069 ± 0.002 | 0.8 ± 0.3 | 4.8 ± 1.6 |
| | 90% N₂ | | | |
| | 10% H₂ | 0.069 ± 0.002 | 0.8 ± 0.3 | 4.7 ± 1.7 |
| | 90% N₂ | | | |
| | 100% Ar | 0.070 ± 0.002 | 1.0 ± 0.3 | 6.0 ± 1.7 |
| CdS:apo-MoFe | 100% N₂ | 0.067 ± 0.002 | 0.6 ± 0.3 | 3.6 ± 1.6 |
| protein | | | | |
| CdS:hydrogenase | 100% N₂ | 0.068 ± 0.002 | 0.9 ± 0.3 | 5.3 ± 2.0 |
| (illuminated) | | | | |
| CdS:hydrogenase | 100% N₂ | 0.068 ± 0.002 | 0.8 ± 0.4 | 5.0 ± 2.1 |
| (dark) | | | | |
| Assay Blank | 100% N₂ | 0.061 ± 0.002 | 0.01 ± 0.2 | 0.1 ± 1.4 |

^{a}Results are the means of N = 4 independent reactions (±SD). CdS:hydrogenase reaction were
performed with [FeFe]-hydrogenase I from Clostridium acetobutylicum, previously shown to
form biohybrids with CdS and to photocatalyze H₂ evolution and are used here as a negative
control for photocatalytic NH₃ production. Reactions with the MoFe protein alone did not
produce any detectable N₂ reduction activity.
^{b}Mean A₅₇₀ values of N = 4 independent reactions (±SD) measured after 2 h of illumination for
a 50 μl aliquot of the 300 μl reaction.
^{c}Calculated from conversion of A₅₇₀ values to a linear fit of the standard plot for NH₄Cl in FIG. 4,
panel A. The linear fit equation, y = a * x + b, where a = 0.0091 ± 0.0002, and b = 0.061 ± 0.001.
Value shown is for a 50 μl aliquot of a 300 μl reaction. N = 4 independent reactions (±SD).
^{d}Total nmol of NH₃ for a 300 μl reaction for each condition (Total nmol in each 300 μl reaction =
nmol in 50 μl aliquot × 6). The total nmol NH₃ was used to calculate rate values shown in FIG.
2. Mean of N = 4 independent reactions (±SD).

Table 4 depicts average raw fluorescence measurements for photochemical NH₃ production by CdS:MoFe protein biohybrids measured by the o-phthalaldehyde fluorescence assay.

TABLE 4

| Sample | ^{a}Fluorescence @ 472 nm |
|---|---|
| CdS:MoFe protein | 165.28 ± 57.05 |
| CdS:apo-MoFe protein | 77.18 ± 13.31 |
| CdS:MoFe protein (dark) | 10.98 ± 29.37 |

^{a}Mean of N = 4 independent samples, ± SD.

Table 5 depicts results of NH₃ and H₂ production by CdS:MoFe protein biohybrids in reactions that are lacking a specific component.

TABLE 5

| | | | ^{c}mol NH₃ | ^{c}nmol NH₃ | mol H₂ mol | nmol H₂ |
|---|---|---|---|---|---|---|
| | | ^{b}Total | mol MoFe | mg MoFe | MoFe | mg MoFe |
| | Absorbance | nmol NH₃ | protein⁻¹ | protein⁻¹ | protein⁻¹ | protein⁻¹ |
| ^{a}Sample | 570 nm | produced | min⁻¹ | min⁻¹ | min⁻¹ | min⁻¹ |
| Complete | 0.136 ± 0.005 | 48.7 ± 2.9 | 81.2 ± 4.8 | 340 ± 20 | 752 ± 75 | 3146 ± 313 |
| (MoFe protein, | | | | | | |
| CdS, light, | | | | | | |
| HEPES) | | | | | | |
| HEPES | 0.068 ± 0.005 | 4.3 ± 1.3 | 7.1 ± 2.2 | 29.8 ± 9 | 2.5 ± 1.0 | 10.4 ± 4.2 |
| CdS | 0.070 ± 0.003 | 5.5 ± 2.3 | 9.1 ± 3.8 | 38.2 ± 16.0 | 1.5 ± 0.5 | 6.3 ± 2.1 |
| Light | 0.069 ± 0.003 | 5.2 ± 1.9 | 8.6 ± 3.2 | 36.1 ± 13.2 | 1.7 ± 0.5 | 6.9 ± 2.1 |
| MoFe protein | 0.062 ± 0.001 | 0.2 ± 0.9 | ^{d}0.3 ± 1.5 | — | ^{d}319 ± 43 | — |
| FeMo-co | 0.067 ± 0.002 | 3.6 ± 1.6 | 6.0 ± 2.7 | 24.9 ± 11.1 | 46 ± 5 | 193 ± 22 |
| (apo-MoFe | | | | | | |
| protein) | | | | | | |

^{a}Reactions were stirred under illumination with 405 nm diode light at 3.5 mW cm⁻². The amount
of NH₃ and H₂ were measured after 2 h.
^{b}Values were calculated using A₅₇₀ values from FIG. 4 (panel A) for a 50 μl aliquot of a 300 μl
reaction. The A₅₇₀ value was fit to the linear equation, y = a * x + b, where a = 0.0091 ± 0.0002,
and b = 0.061 ± 0.001 to obtain the value in nmol of NH₃ in 50 μl, and multiplied by 6 to obtain
the total NH₃ produced in the 300 μl reaction. Mean of N = 4 independent reactions (±SD).
^{c}NH₃ levels were measured by the Biovision colorimetric assay and are not corrected for the
background from apo-MoFe reactions. Background corrected turnover numbers are listed in
Table 10.
^{d}Normalized as nmol product nmol⁻¹ CdS.

Table 6 depicts a comparison of NH₃ and H₂ production rates by nitrogenase (MoFe protein:Fe protein) and CdS:MoFe protein biohybrids under optimized conditions for each of the two reactions.

TABLE 6

| | mol NH₃ | nmol NH₃ | mol H₂ | nmol H₂ |
|---|---|---|---|---|
| | (mol MoFe | (mg MoFe | (mol MoFe | (mg MoFe |
| | protein)⁻¹ | protein)⁻¹ | protein)⁻¹ | protein)⁻¹ |
| Sample | min⁻¹ | min⁻¹ | min⁻¹ | min⁻¹ |
| ^{a}MoFe | 119 | 500 | 460 | 1932 |
| protein:Fe | | | | |
| Protein + ATP | | | | |
| ^{b}CdS:MoFe | 75.2 ± 6.2 | 314 ± 47 | 729 ± 76 | 3037 ± 317 |
| protein | | | | |
| biohybrids | | | | |

^{a}Reactions consisted of 0.1 mg MoFe protein, 0.5 mg Fe protein and ATP under 100% N₂ at 30° C.
The NH₃ produced was measured by the fluorescence assay.
^{b}Reactions were conducted as described in materials and methods. NH₃ was measured using the
colorimetric assay. Mean of N = 4 independent reactions, ± SD. Values are corrected for non-
catalytic background levels of NH₃ measured in CdS:apo-MoFe protein samples listed in Table 5.

Table 7 depicts parameters used to estimate the quantum yield of product formation from N₂ reduction by CdS:MoFe protein biohybrids.

TABLE 7

| Parameter | Value |
|---|---|
| Lamp output (405 nm) | 34 ± 7 mW |
| ^{a}Light power at sample | 1.8 ± 0.4 mW |
| ^{b}Incident photon rate | 3.6 ± 0.7 × 10⁻⁷ mol min⁻¹ |
| ^{c}Total incident photon | 4.3 ± 1 × 10⁻⁵ mol |
| ^{d}Photons absorbed | 4.3 ± 0.9 × 10⁻⁶ mol |

^{a}Light power at sample = lamp output x (sample illumination area ÷ output illumination area) =
34 mW × (0.5 cm² ÷ 9.5 cm²) = 1.78 ± 0.40 mW.
^{b}Calculated based on photon wavelength = 405 nm with an energy/photon = 4.9 × 10⁻¹⁹ J.
^{c}Calculated for 120 min of illumination time.
^{d}Photons absorbed was determined based on the CdS:MoFe protein reaction having a
transmittance of 89% at 405 nm, to obtain the photons absorbed as 11%. (4.3 ± 1 × 10⁻⁵ incident
photons × 11%) = 4.3 ± 0.9 × 10⁻⁶ photons absorbed.

Table 8 depicts the electron requirement for NH₃ and H₂ product formation at 2 h illumination and estimated quantum yield by CdS:MoFe protein biohybrids from N₂ reduction.

TABLE 8

| | | ^{b}Electrons required | Photons | ^{c}Estimated quantum |
|---|---|---|---|---|
| | ^{a}Amount | for product | absorbed | yield of product |
| Product | (nmol) | formation (mol) | (mol) | formation (%) |
| NH₃ | 45 ± 7 | 0.14 ± 0.02 × 10⁻⁶ | 4.3 ± 0.9 × | 3.3 ± 0.8 |
| | | | 10⁻⁶ | |
| H₂ | 437 ± 45 | 0.87 ± 0.09 × 10⁻⁶ | 4.3 ± 0.9 × | 20.2 ± 5 |
| | | | 10⁻⁶ | |
| NH₃ + | 482 ± 46 | 1.01 ± 0.09 × 10⁻⁶ | 4.3 ± 0.9 × | 23.5 ± 5 |
| H₂ | | | 10⁻⁶ | |

^{a}Mean of N = 4 independent reactions (±SD) after 2 h of illumination. The product values are
corrected for background from CdS:apo-MoFe protein reactions.
^{b}nmol electrons required per product:
½N₂ + 3H⁺ + 3e⁻ → NH₃
2H₂ + 2e⁻ → H₂.
Total nmol e⁻ based on total products after 120 min = (45 nmol NH₃ × 3e⁻) + (437 nmol H₂ × 2e⁻) = 1009 nmol e⁻.
^{c}Quantum Yield = (mol e⁻ used in product formation) ÷ (mol of absorbed photons) × 100%. The
observed product ratio for CdS:MoFe protein catalyzed N₂ reduction is ~1 mol NH₃ to 10 mol
H₂, which requires [(1 × 3e⁻) + (10 × 2e⁻)] = 23 e⁻.
The number of absorbed photons (n) required to provide CdS:MoFe protein biohybrid with 23 e⁻
is equal to 23 e⁻ ÷ 1/QY, or 23 ÷ 0.235 = 98. Thus, n = 98 absorbed photons.

Table 9 depicts uncorrected NH₃ production time course data for CdS:apo-MoFe protein and CdS:MoFe protein biohybrids under illumination (FIG. 2, inset).

TABLE 9

| | | Total | mol NH₃ | mol NH₃ |
|---|---|---|---|---|
| | ^{a}Total | nmol NH₃ | (mol MoFe | (mol MoFe |
| | nmol NH₃ | CdS:apo- | protein)⁻¹ | protein)⁻¹ CdS: |
| Illumination | CdS:MoFe | MoFe | CdS:MoFe | apo-MoFe |
| time (min) | protein | protein | protein | protein |
| 20 | 5.4 ± 1.9 | 1.9 ± 0.5 | 1075 ± 388 | 383 ± 96 |
| 40 | 10.3 ± 1.6 | 3.5 ± 0.9 | 2061 ± 314 | 700 ± 175 |
| 60 | 20.8 ± 2.8 | 4.2 ± 1.0 | 4137 ± 562 | 827 ± 207 |
| 90 | 39.2 ± 4.7 | 7.4 ± 1.9 | 7814 ± 943 | 1479 ± 371 |
| 120 | 48.9 ± 6.7 | 3.6 ± 2.2 | 9740 ± 133 | 719 ± 438 |
| 210 | 58.1 ± 8.1 | 5.5 ± 1.4 | 11573 ± 1607 | 1098 ± 275 |
| 300 | 64.2 ± 8.3 | 8.8 ± 2.2 | 12795 ± 1645 | 1760 ± 438 |

^{a}Mean of N = 4 independent measurements, ± SD.

Table 10 depicts Background corrected N₂ reduction/NH₃ production time course data for CdS:MoFe protein biohybrids under illumination (FIG. 2, inset).

TABLE 10

| Illumination | ^{a}mol NH₃ |
|---|---|
| time (min) | (mol MoFe protein)⁻¹ |
| 20 | 692 ± 399 |
| 40 | 1361 ± 360 |
| 60 | 3310 ± 599 |
| 90 | 6335 ± 1013 |
| 120 | 9021 ± 1403 |
| 210 | 10475 ± 1631 |
| 300 | 11036 ± 1702 |

^{a}Values are corrected for non-catalytic background levels of NH₃ measured in CdS:apo-MoFe
protein samples listed in Table 5. Error calculated by standard error propagation methods using
sample error and CdS:apo-MoFe reaction error (σ_{TOF} = \sqrt{σₛₐₘₚₗₑ² + σ_{Apo-MoFe protein}²}).
Mean of N = 4 measurements, ± SD.

Table 11 depicts Inhibition of Fe protein/ATP dependent H₂ production by MoFe protein in the presence of CdS.

TABLE 11

| Sample | nmol H₂ (mg MoFe protein)⁻¹ min⁻¹ |
|---|---|
| ^{a}MoFe protein + Fe Protein/ATP | 1961 ± 192 |
| ^{b}CdS:MoFe protein biohybrids + | 185 ± 50 |
| Fe Protein/ATP | |

^{a}Reactions consisted of 0.1 mg MoFe protein, 0.5 mg Fe protein and ATP under 100% N₂ at 30° C.,
in the dark, and in a buffer composed of 30 mM phosphocreatine, 5 mM ATP, 0.2 mg/mL
creatine phosphokinase, and 1.2 mg/mL BSA) in 100 mM HEPES buffer at pH 7.0. The nmol of
H₂ was measured by GC. Mean of N = 4 independent reactions, ± SD.
^{b}CdS:MoFe protein biohybrids; 16.7 nM CdS, 16.7 nM MoFe protein.

### Effect of MoFe Protein Inhibitors on Photocatalytic N₂ Reduction

Samples of CdS:MoFe protein were prepared as described above in 100% N₂ atmosphere. The sample headspace was then equilibrated under 100% argon, or 10% acetylene, CO or H₂ and 90% N₂ prior to illumination. Solutions were stirred under illumination with 405 nm diode light (3.5 mW cm⁻² at the sample) in sealed vials. The total amount of NH₃ and H₂ produced were measured as described above.

Experiments using known inhibitors of Mo-dependent nitrogenase activity indicate that the N₂ reduction reaction occurs at catalytic site FeMo cofactor (FeMo-co) of the MoFe protein. Acetylene (C₂H₂), carbon monoxide (CO) and H₂ are all known to specifically inhibit the N₂ reduction reaction at FeMo-co. Acetylene acts as a substrate to inhibit N₂ and proton reduction at FeMo-co. In contrast, CO is known to inhibit N₂ reduction by blocking the N₂ binding site at FeMo-co, but proton reduction to H₂ is unaffected.

The addition of either H₂, CO or C₂H₂ at 10% to a 90% N₂ gas phase decreased the N₂ reduction rates by CdS:MoFe protein biohybrids to the background levels observed with apo-MoFe protein (FIG. 2; Tables 12 and 13). The results are consistent with the effect of these inhibitors on preventing MoFe protein catalysis in the Fe protein, ATP-driven physiological reaction. Photochemical H₂ production by CdS:MoFe protein biohybrids was also inhibited by 10% C₂H₂, but only slightly decreased under 10% CO compared to rates under 100% N₂ (FIG. 5). Consistent with N₂ being a substrate of CdS:MoFe protein biohybrids, the rates of H₂ production were 25% higher when N₂ was replaced with 100% argon (FIG. 5). Together, the inhibition results are consistent with photocatalysis by CdS:MoFe protein biohybrids occurring at the FeMo-co site of the MoFe protein by a mechanism that is similar to the Fe protein, ATP-coupled reaction.

Table 12 depicts data used to determine the effects of gaseous inhibitors on TOF of NH₃ production plotted in FIG. 2, uncorrected for non-catalytic background levels of NH₃ measured in CdS:apo-MoFe protein samples.

TABLE 12

| | Gas | ^{a}nmol | ^{a}Total | |
|---|---|---|---|---|
| | phase of | NH₃ | nmol NH₃ | ^{a}TOF |
| Sample | reaction | detected | produced | (min⁻¹) |
| CdS:MoFe | 100% N₂ | 8.2 ± 0.6 | 48.9 ± 3.4 | 81.2 ± 5.6 |
| protein | | | | |
| | 10% C₂H₂, | 0.9 ± 0.3 | 5.2 ± 1.6 | 8.7 ± 2.7 |
| | 90% N₂ | | | |
| | 10% CO, | 0.8 ± 0.3 | 4.8 ± 1.6 | 8.0 ± 2.7 |
| | 90% N₂ | | | |
| | 10% H₂, | 0.8 ± 0.3 | 4.7 ± 1.7 | 7.8 ± 2.8 |
| | 90% N₂ | | | |
| | 100% Ar | 1.0 ± 0.3 | 6.0 ± 1.7 | 9.9 ± 2.8 |
| CdS:apo-MoFe | 100% N₂ | 0.6 ± 0.3 | 3.6 ± 1.6 | 6.0 ± 2.6 |
| protein | | | | |

^{a}Mean of N = 4 independent measurements, ± SD.

Table 13 depicts TOF of NH₃ production by CdS:MoFe protein plotted in FIG. 2, and corrected for non-catalytic background levels of NH₃ measured in CdS:apo-MoFe protein samples.

TABLE 13

| Gas phase of | ^{a}Absorbance | ^{b}nmol NH₃ | ^{c}nmol NH₃ | ^{d}Corrected |
|---|---|---|---|---|
| reaction | 570 nm | detected | produced | TOF (min⁻¹) |
| 100% N₂ | 0.136 ± 0.005 | 8.2 ± 0.6 | 48.9 ± 3.4 | 75.2 ± 6.2 |
| 10% C₂H₂, 90% N₂ | 0.069 ± 0.002 | 0.9 ± 0.3 | 5.2 ± 1.6 | 2.7 ± 3.7 |
| 10% CO, 90% N₂ | 0.069 ± 0.002 | 0.8 ± 0.3 | 4.8 ± 1.6 | 2.1 ± 3.8 |
| 10% H₂, 90% N₂ | 0.069 ± 0.002 | 0.8 ± 0.3 | 4.7 ± 1.7 | 1.9 ± 3.8 |
| 100% Ar | 0.070 ± 0.002 | 1.0 ± 0.3 | 6.0 ± 1.7 | 3.9 ± 3.8 |

^{a}Mean of N = 4 independent reactions after 2 h of illumination.
^{b}Calculated using A₅₇₀ values for a 50 μl aliquot of a 300 μl reaction fit to the plot in FIG. 4,
panel A. The A₅₇₀ value was fit to the linear equation, y = a * x + b, where a = 0.0091 ± 0.0002,
and b = 0.061 ± 0.001 to obtain the value in nmol of NH₃ in 50 μl.
^{c}Calculated by multiplying amount of the NH₃ detected in a 50 ul aliquot by 6 to obtain the total
NH₃ produced in the 300 μl reaction.
^{d}Calculated by subtracting CdS:apo-MoFe protein sample background (3.6 ± 1.6 nmol) from the
total nmol produced; Mean of N = 4 independent reactions (±SD). SD was calculated by standard
error propagation method using sample error and CdS:apo-MoFe protein sample error
(σ_{TOF} =\sqrt{σₛₐₘₚₗₑ² + σ_{Apo-MoFe protein}²}).

### Fluorescence Assay of NH₃ Production

Ammonia production was verified by a second, independent method of ammonia detection based on fluorescence detection using o-phthaladehyde. CdS nanorods demonstrate a quenching effect on the fluorescence of this assay, so they were removed before the assay. After illumination, the samples were run through a 10 kDa spin concentrator (Corning Spin-X UF) at 14,000 rpm for 5 minutes to separate CdS:MoFe protein biohybrids. Fifty μL of the flow through was added to 1 mL of a solution of 20 mM o-phthalaldehyde, 0.2 M phosphate buffer (pH 7.3), 5% ethanol, 3.4 mM β-mercaptoethanol. Samples were incubated in the dark for 30 minutes at room temperature. The fluorescence (λexcitation/λemission=410 nm/472 nm) of the solutions was measured using a Shimadzu Model RF-5301 PC spectrofluorometer. A calibration curve was created by preparing a solution of CdS:MoFe protein biohybrids (16.67 nM) in assay buffer, incubating it in the dark for 90 minutes, then running it through a 10 kDa spin concentrator. Ammonium chloride was then added, in appropriate amounts, to aliquots of the filtered solution to a final volume of 50 μL then reacted, incubated, and assayed as described above (FIG. 4, panel B). Ammonia production above background levels was in agreement with the results of the colorimetric assay.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

Other objects, advantages, and novel features of the present invention will become apparent from the following detailed description of the invention when considered in conjunction with the accompanying drawings.

What is claimed is:

1. A method of producing ammonia, comprising
a) contacting a nitrogenase biohybrid complex with nitrogen;
b) exposing the nitrogenase biohybrid complex to light to generate ammonia; and
c) isolating the generated ammonia.

2. The method of claim 1, wherein the light has a wavelength from about 380 nm to about 450 nm.

3. The method of claim 1, wherein the intensity of the light at the biohybrid complex is from about 1.8 mW cm⁻² to about 25 mW cm⁻².

4. The method of claim 1, wherein the biohybrid complex comprises CdS nanoparticles.

5. The method of claim 1, wherein the isolated ammonia is about 86 mol NH₃ mol biohybrid complex⁻¹ min⁻¹.

6. The method of claim 1, wherein the isolated ammonia is about 12000 mol NH₃ mol biohybrid complex⁻¹ after about 300 minutes of exposure to light.
